(19) United States
(12) Patent Application Publication
(10) Pub. No.: US 2006/0199209 A1
(43) Pub. Date: Sep. 7, 2006

(54) Enhanced detection of biological and bioactive components by resonance Raman spectroscopy

(76) Inventor: Neal Arthur Siegel, Morris, IL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/366,435

(22) Filed: Mar. 3, 2006

Related U.S. Application Data
(60) Provisional application No. 60/659,027, filed on Mar. 3, 2005.

(51) Int. Cl. C12Q 1/68; G01N 33/53; G01N 33/554; G01N 33/569

(52) U.S. Cl. 435/6; 435/7.1; 435/7.32

(57) ABSTRACT

A method for the detection of compounds, including biological macromolecules and cells, using changes in Raman spectroscopic properties upon binding of at least one analyte binding partner. The method comprises contacting at least one analyte with at least one analyte binding partner, binding at least one analyte to at least one analyte binding partner to form a complex, and detecting the complex using the change in Raman light scattering.

This application claims the benefit of U.S. Provisional Application No. 60/659,027, filed Mar. 3, 2005.

The present invention is directed to identifying by optical detection biologically active compounds monitored through the use or application of resonance Raman spectroscopy in an aqueous sample. Those skilled in the art will recognize the broader application to other physical measurement techniques including, but not limited to, spectrophotometric and spectroscopic techniques such as fluorescence, fluorescence correlation, fluorescence polarization, electron spin resonance (electron paramagnetic resonance), nuclear magnetic resonance, circular dichroism, magnetic circular dichroism, light transmission, absorbance and reflectance, chemiluminescence and the like.

Resonance Raman spectroscopy has been described, for example, in U.S. Pat. No. 4,847,198, as a method for identifying bioorganisms through the spectral qualities of intrinsic organism components such as DNA and proteins. The basic technique consists of the irradiation of whole organisms with ultraviolet laser light at a wavelength selected to minimize intrinsic fluorescence and through various detection means develop spectra of resonating, Raman scattered light. These spectra are highly complex wavelength shift patterns that relate to the biological molecules and structures of the irradiated organisms. See, e.g., Fodor et al., J. Am. Chem. Soc., 111(15), 1731 (1989) (“Deep-Ultraviolet Raman Excitation Profiles and Vibronic Scattering Mechanisms of Phenylalanine, Tyrosine, and Tryptophan”); and Cheng et al., Biophysical J., 83, 502-509 (2002) (“Laser-Scattering Coherent Anti-Stokes Raman Scattering Microscopy and Applications to Cell Biology”).

The complexity of the resonance Raman signal coupled with the sensitivity of the technique to biological components makes interpretation of the reported spectra difficult and impractical. For biological assay, both sensitivity and specificity are necessary qualities for a technique to have diagnostic value. Without sensitivity and specificity, complex results are virtually uninterpretable and provide small value for the practitioner.

Medical diagnostics utilize immunochemical techniques to provide specificity in the detection of biologically active components of a sample. Antibodies developed to specific compounds are known to have high affinity for these components. Antibodies alone generally provide limited detectability and as such are typically chemically modified with labels or tags that serve to report on the location and or concentration of the antibody during a reaction in a sample. Various techniques may be applied to the measurement of these types of systems including radioactive or enzymatic labeling, fluorescence and other optical techniques. See, e.g., The Immunoassay Handbook, David Wild (ed), Stockton Press (1994) (ISBN 1-56159-064-9).

Various spectroscopic techniques such as fluorescence, circular dichroism, and electron spin resonance have traditionally been applied to the study of biological component structure such as those found in proteins. In fact, these techniques can often be used to study real-time, allosteric changes and imposed structural effects during enzymatic activity, component binding and other physical processes that alter the molecular movement or shape. Siegel et al., Biochimica et Biophysics Acta, 744, 36-45 (1983) (“Aluminum Interaction with Calmodulin: Evidence for Altered Structure and Function from Optical and Enzymatic Studies”). Resonance Raman has been demonstrated to have utility in bio-organic structural and physical chemistry studies as already discussed.

It is known from the literature that resonant Raman scattering is due to specific structural organization when used with biological materials such as DNA and proteins. From various studies and knowing the energy of the scattered light, estimates can be made of the structural detail from which the resonant scattered light emerges. See, e.g., Balakrishnan et al., J. Mol. Biol., 340, 857-868 (2004) (“Hemoglobin Site-mutants Reveal Dynamical Role of Interhelical H-bonds in the Allosteric Pathway: Time-resolved UV Resonance Raman Evidence for Intra-dimer coupling”). In the case of a newly formed structure, the resonant energy should be evident through the formation of a new peak or peaks or integrated onto an existing one(s) of similar or identical energy. Thus, a resonance Raman spectrum properly analyzed will provide identification of the content of the system that includes the newly formed adduct.

One important factor to consider is the wavelength of the exciting light. Since the light is partially absorbed by the resonating biomolecule, different wavelengths will “report” information about the molecules with which they interact. Resonant structures scatter light in a manner described as the Raman effect. The frequency of this scattered tight is correlated with the energy level of the atomic structure with which it has interacted. This supplies information regarding the molecule giving rise to the Raman signal. When a biological structure interacts to scatter the light in this way, the signal becomes a reporter of the specific structure and hence, may be considered to be a “fingerprint” of the molecule.

The present invention describes the application of resonance Raman spectroscopy to monitor specific adduct formation as found, for example in an antibody binding to its target molecule. The binding of two biologically active components creates a new combined molecule with unique structural characteristics. The potential use of resonance Raman spectroscopy to report this new structure has the benefit of reducing the combined, complex signals typically found to background noise for the newly created, “engineered” signal. A benefit to the present invention is that the chemical structure of the newly formed adduct is actually an engineered feature that is uniquely traceable and reportable. This obviates the need for complicated subtractive or comparative signal processing techniques since the signal is newly formed. Signal formation may be followed through a time sequence during its formation that relates to the binding reaction of the actual components in the sample mixture. Alternately, the endpoint of the binding reaction may be quantified for the mixture. These effects in combination lend themselves to the ability to calibrate the signal for further refinement to the method beyond identification into quantitation.

The present enhanced detection technology is a novel improvement on traditional biological component labeling and identification technology, and overcomes their limitations, principally low specificity and quantitation, by relying on at least one of the following parameters:

1. Using binding partners having high specificity for targets of interest in biological assays. These binding partners may be naturally occurring, bioengineered or artificially produced antibodies, binding protein, transport proteins, enzymes, DNA primers, RNA primers, cDNA sequences, DNA sequences, RNA sequences, feature selective dyes or molecules, proteins, peptides, nanoparticles with or without chemical modifications to render or increase their structural detectability upon adduct formation.

2. Using a sensitive detection method in combination with the specific binding partners to detect structural changes associated with the binding event of interest. The binding event may be a sequentially occurring event over time for which the kinetic changes are the quantified feature or an endpoint event for which the magnitude of the signal is detected at a time where the reaction has completed or has reached a stable or equilibrium point. Alternately, the reaction endpoint signal may be used as quantification of the reaction.

3. Using a correlation analytical technique in conjunction with the adduct formation to monitor movement or state changes of the adduct within a biological matrix such as a cell membrane or in a defined space through which the reporter molecule or molecules transverse. Examples of such correlation techniques include, but are not limited to, fluorescence recovery after photobleaching (FRAP), fluorescence correlation spectroscopy (FCS), the transition of a suspension of solid particles in a liquid (sol) to an apparent solid, jelly-like material (gel) (sol:gel transition), and tc calculations.

4. Using resonance Raman spectroscopy as the preferred optical methods for implementation of the enhanced adduct detection technique. Resonance Raman as a technique offers advantages over other techniques due to at least one of its increased signal to noise ratio, simple output format and selectability of input wavelengths to avoid common interferences from intrinsic fluorescence and similar physical measurement issues.

In one embodiment of the invention, at least one analyte is detected by contacting at least one analyte with at least one analyte binding partner, binding at least one analyte to at least one analyte binding partner to form a complex, and detecting the complex using the change in Raman light scattering wherein the detection does not rely upon surface enhanced Raman light scattering.

In another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in Raman light scattering.

In yet another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in Raman light scattering wherein the detection does not rely upon surface enhanced Raman light scattering.

In a further embodiment of the invention, at least one analyte is detected by contacting at least one analyte with at least one analyte binding partner, binding at least one analyte to at least one analyte binding partner to form a complex, and detecting the complex using the change in resonant Raman light scattering wherein the detection does not rely upon surface enhanced Raman light scattering.

In another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in resonant Raman light scattering.

In yet another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in resonant Raman light scattering wherein the detection does not rely upon surface enhanced Raman light scattering.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a representative Raman spectrum Listeria innocua bacteria of the type collected with shift numbers (cm⁻¹) plotted on the abscissa and signal magnitude plotted on the ordinate (arbitrary units) marked at 250.6 cm⁻¹ (a), 473.0 cm⁻¹ (b), 514.8 cm⁻¹ (c), 559.2 cm⁻¹ (d), 816.1 cm⁻¹ (e), 872.6 cm⁻¹ (f), 1,052.2 cm⁻¹ (g), and 1,065.6 cm⁻¹ (h).

FIG. 2 illustrates typical results of the experiment plotting Raman intensity for the 250 cm⁻¹ peak in the presence (+AB₃) and absence (−Ab) of antibody at 0, 100, 1,000, and 100,000 CFU of Listeria innocua bacteria; measuring Raman intensities of 415.34, 405.51, 1,003.84, and 1,524.96, in the presence (+AB₃) of antibody and 415.34, 1,549.24, 1,328.88, 1,387.71, in the absence (−Ab) of antibody at 0, 100, 1,000, and 100,000 CFU respectively

FIG. 3 illustrates the effect of increasing antibody concentration on Raman intensity at 250 cm⁻¹ in the absence of bacteria with intensities of 415.34, 268.47, 242.06, and 150.14, measured using the 0 mg/ml (LOA0), 1 mg/ml (LOA1), 10 mg/ml (LOA2), and 100 mg/ml (LOA3) dilutions of Listeria innocua antibody.

FIG. 4 illustrates the effect of antibody concentration and bacterial concentration on Raman intensity at 860 cm⁻¹ with intensities measured using the 0 mg/ml (LOA0), 1 mg/ml (LOA1), 10 mg/ml (LOA2), and 100 mg/ml (LOA3) dilutions of Listeria innocua antibody at 0, 100, 1,000, and 100,000 CFU of of Listeria innocua bacteria.

FIG. 5 illustrates the calculated ratio of Raman intensity at 250 cm⁻¹ over Raman intensity at 860 cm⁻¹ in the presence (+AB₃) and absence (−Ab) of antibody at 0, 100, 1,000, and 100,000 CFU of Listeria innocua bacteria; calculating Raman intensities ratios (250 cm⁻¹ /860 cm⁻¹) of 1.0, 6.1, 4.7, and 8.3, in the presence (+AB₃) of antibody and 1.0, 2.2, 5.5, and 7.4, in the absence (−Ab) of antibody at 0, 100, 1,000, and 100,000 CFU respectively.

### DESCRIPTION OF THE EMBODIMENTS

In one embodiment of the invention, at least one analyte is detected by contacting at least one analyte with at least one analyte binding partner, binding at least one analyte to at least one analyte binding partner to form a complex, and detecting the complex using the change in Raman light scattering wherein the detection does not rely upon surface enhanced Raman light scattering.

In another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in Raman light scattering.

In yet another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in Raman light scattering wherein the detection does not rely upon surface enhanced Raman light scattering.

In a further embodiment of the invention, at least one analyte is detected by contacting at least one analyte with at least one analyte binding partner, binding at least one analyte to at least one analyte binding partner to form a complex, and detecting the complex using the change in resonant Raman light scattering wherein the detection does not rely upon surface enhanced Raman light scattering.

In another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in resonant Raman light scattering.

In yet another embodiment of the invention, at least one analyte is detected by contacting at least one analyte with at least one analyte binding partner, binding at least one analyte to at least one analyte binding partner to form a complex, wherein at least one unbound binding partner is not separated from at least one binding partner bound in a complex, and detecting the complex using the change in resonant Raman light scattering wherein the detection does not rely upon surface enhanced Raman light scattering.

For each of the above embodiments the complex may be detected using resonant Raman light scattering.

For each of the above embodiments the presence of the complex may be detected using a Raman signal measured at a single wavenumber. For example, the Raman signal at 250 cm⁻¹ can be used to detect the presence of the complex. Similarly, the Raman signal at 860 cm⁻¹ can be used to detect the presence of the complex.

Similarly, the presence of the complex may be detected using Raman signals measured at more than one wavenumber. For example, the Raman signals at 250 cm⁻¹ and 860 cm⁻¹ can be used to detect the presence of the complex.

A variety of molecules can serve as binding partners in the above embodiments. In one embodiment, proteins, peptides, naturally occurring antibodies, bioengineered antibodies, artificially produced antibodies, binding proteins, transport proteins, and enzymes may serve as binding partners. For example, the Goat anti Listeria innocua antibody can serve as the binding partner used in the detection Listeria innocua according to the methods of this application.

In another embodiment, DNA primers, RNA primers, cDNA sequences, DNA sequences, and RNA sequences may serve as binding partners.

In an alternate embodiment, feature selective dyes and feature selective molecules may serve as binding partners.

In another embodiment, nanoparticles with or without chemical modifications to render or increase their structural detectability upon adduct formation may serve as binding partners.

The method may be used to detect a variety of analytes. In one embodiment the analyte is a molecule. In another embodiment the analyte is a biological macromolecule.

In an alternate embodiment the analyte is a cell. In yet another embodiment the analyte is a bacteria. In an alternate embodiment the analyte is a pathological bacteria. For example, the bacteria may be Listeria monocytogenes, Salmonella spp., Clostridium botulinum, Staphylococcus aureus, Campylobacter jejuni, Yersinia enterocolitica, Yersinia pseudotuberculosis, Vibrio cholerae O1, Vibrio cholerae non-O1, Vibrio parahaemolyticus, Vibrio vulnificus, other vibrios, Clostridium perfringens, Clostridium perfringens, Bacillus cereus, Aeromonas hydrophila and other spp., Plesiomonas shigelloides, Shigella spp., Streptococcus, Enterovirulent Escherichia Coli Group (EEC Group), Escherichia coli—enterotoxigenic (ETEC), Escherichia coli—enteropathogenic (EPEC), Escherichia coli O157:H7 enterohemorrhagic (EHEC), or Escherichia coli—enteroinvasive (EIEC).

Another embodiment includes either a liquid or swabbed sample being mixed or eluted into a solution containing antibodies directed at an organism or target of interest. The signal may then be compared to a calibrated response to indicate the presence and quantity of the target of interest. Background subtraction may be used to further enhance the detection of the signal.

### EXAMPLE

The following provides an example of the ability to quantify the presence of a bacteria with antibody. The materials used were Listeria innocua at three concentrations (Colony Forming Units; CFU) and polyclonal antibody reactive with a wide group of Listeria subspecies. The resonance Raman system used an excitation wavelength of 229 nm at 2 mW power. Data were collected using a CCD array. Spectral peak heights and counts were extracted using Gram's Al program (Thermo Galactic Gram's/Al version 7.00©1 991-2002). Data were tabulated and plotted in Microsoft Excel™ 2000.

Materials and Methods

Laser Source: The second harmonic of a Q-switched Nd:YLF laser operating at 1 kHz is used to pump a Ti:sapphire (Ti:S) laser, which gives tunable output between 770 nm and 940 nm. The Ti:S laser output is frequency doubled and tripled and or quadrupled to generate visible and UV wavelengths. We have access to 195-235 nm and 257-280 nm in the UV region and 385-470 nm in the visible region.

Spectrometer: Spex 1269, (1.26 meter) equipped 3600 grooves/mm grating blazed at 250 nm. The spectral bandpass of the instrument is ˜5.5 nm. CCD used as the detector. CCD has 1340 (spectral axis)×400 (intensity axis) pixels.

Antibody: Goat anti Listeria affin. pure, catalog # 70-XG19; 5.7 mg/ml; 200 mg, Lot # X0092202, from Fitzgerald Industries International, Inc. Listeria Innocua: Marshfield Clinic Laboratories-Food Safety Services.

Plate Counts

Target=200,000 ([Li]₃)

Actual=235,000; 189,000; 260,000; 167,000; 210,000

Average=212, 200

Target=2000 ([Li]₂)

Actual=8750; 6400: 10240: 8960: 7680;

Average=8406

Target=200 ([Li]₁)

Actual=291; 286; 368; 265; 317

Average=305

Antibody dilutions were made using Buffered Peptone water (bacterial suspension solution) supplied by Marshfield with Listeria innocua aliquots. Concentrations were [Ab]₁ 1 mg/ml; [Ab]₂ 10 mg/ml; [Ab]₃ 100 mg/ml.

The antibody and Listeria dilutions were mixed 1:1 (to 400 ml total sample volume) for signal detection at 2 mW power for a 229 nm excitation wavelength. Spectra were collected every 30 seconds for 10 minutes. There was no significant difference in scans after 1 minute, the data from the 10 minute scans are used here as examples.

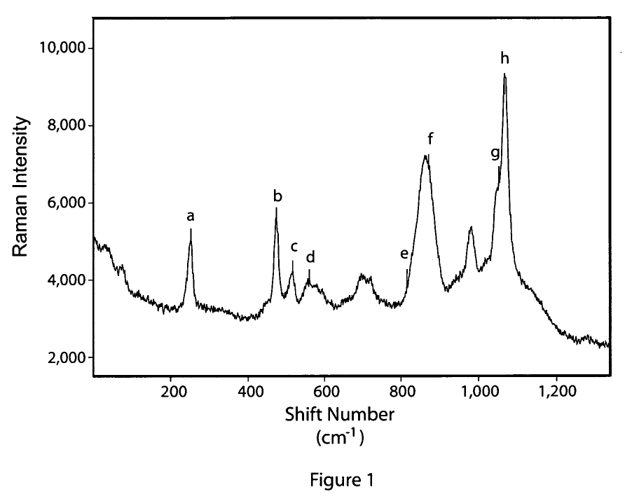

A representative resonance Raman spectrum of the type collected is shown in FIG. 1. Shift numbers (cm⁻¹) are plotted on the abscissa; signal magnitude is plotted on the ordinate (arbitrary units). Peak labels are cm⁻¹.

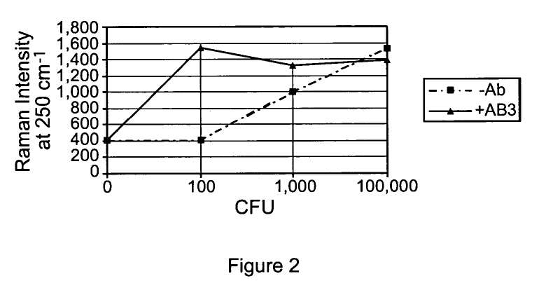

Typical results of the experiment are shown for the 250 cm⁻¹ peak in FIG. 2. Note the difference between the signal for the bacterial solution containing Antibody (+AB3) and the bacterial solution without Antibody (−Ab).

Between 0 and 100 CFU, the signal increase of the solution is almost 4 fold due to the addition of antibody. This translates into the practical application of this technique to increase the sensitivity of detection lower than just the presence of bacteria can exhibit alone without additional chemical labels such as fluorophores or Raman-active chemicals added to the system.

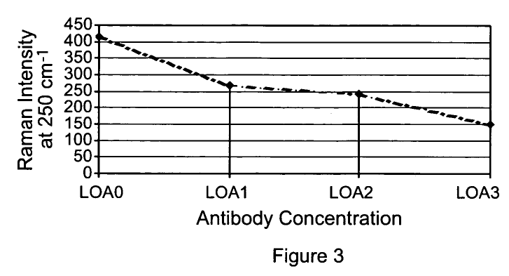

Antibody addition to the solution without bacteria shows a minor signal decrement of the 250 cm⁻¹ peak as noted in the graph shown in FIG. 3.

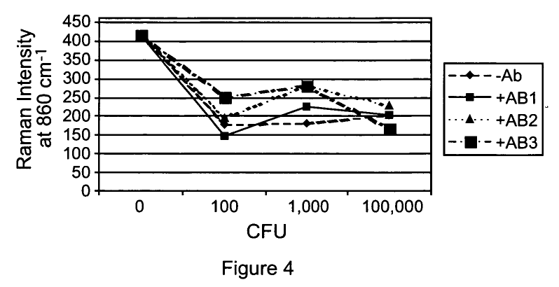

A means of calibration may be demonstrated by the following data in FIG. 4. Use of a peak whose response is not correlated with the dose of bacteria or antibody can establish a normalized response.

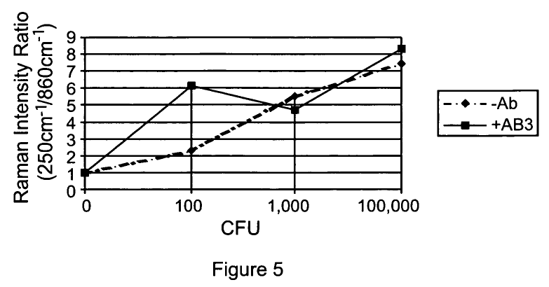

A ratio of the two shift vales may be used to normalize the result if required by the measurement as demonstrated for the 250/860 shifts' ratio in FIG. 5.

These experimental results show that an antibody can react with a bacteria found in a suspension and thus increase the detectability of the organism by resonance Raman spectroscopy by at least an order of magnitude. This indicates that special chemical labeling of the antibody is unnecessary for the application of resonance Raman spectroscopy for the detection of bacteria in a sample for which a specific antibody is added.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for the detection of at least one analyte comprising
a. contacting at least one analyte with at least one analyte binding partner,
b. binding at least one analyte to at least one analyte binding partner to form a complex, wherein at least one unbound binding partner is not separated from at least one binding partner bound in a complex,
c. detecting the complex using the change in Raman light scattering wherein the detection does not rely upon surface enhanced Raman light scattering.

2. The method of claim 1 wherein the complex is detected using resonant Raman light scattering.

3. The method of claim 1 wherein a Raman signal at a single wavenumber is used to detect the presence of the complex.

4. The method of claim 1 wherein a Raman signal at 250 cm⁻¹ is used to detect the presence of the complex.

5. The method of claim 1 wherein a Raman signal at 860 cm⁻¹ is used to detect the presence of the complex.

6. The method of claim 1 wherein Raman signals at more than one wavenumber are used to detect the presence of the complex.

7. The method of claim 1 wherein Raman signals at 250 cm⁻¹ and 860 cm⁻¹ are used to detect the presence of the complex.

8. The method of claim 1 wherein the at least one binding partner is chosen from proteins, peptides, naturally occurring antibodies, bioengineered antibodies, artificially produced antibodies, binding proteins, transport proteins, and enzymes.

9. The method of claim 1 wherein the at least one binding partner is chosen from DNA primers, RNA primers, cDNA sequences, DNA sequences, and RNA sequences.

10. The method of claim 1 wherein the at least one binding partner is chosen from feature selective dyes and feature selective molecules.

11. The method of claim 1 wherein the at least one binding partner is chosen from nanoparticles with or without chemical modifications to render or increase their structural detectability upon adduct formation.

12. The method of claim 1 wherein the at least one analyte is a molecule

13. The method of claim 1 wherein the at least one analyte is a biological macromolecule.

14. The method of claim 1 wherein the at least one analyte is a cell.

15. The method of claim 1 wherein the at least one analyte is a bacteria.

16. The method of claim 1 wherein the at least one analyte is a pathological bacteria.

17. A method for the detection of at least one analyte comprising
a. contacting at least one analyte with at least one analyte binding partner,
b. binding at least one analyte to at least one analyte binding partner to form a complex,
c. detecting the complex using the change in Raman light scattering wherein the detection does not rely upon surface enhanced Raman light scattering.

18. A method for the detection of at least one analyte comprising
a. contacting at least one analyte with at least one analyte binding partner,
b. binding at least one analyte to at least one analyte binding partner to form a complex, wherein at least one unbound binding partner is not separated from at least one binding partner bound in a complex,
c. detecting the complex using the change in Raman light scattering.
